(19) United States
(12) Patent Application Publication
(10) Pub. No.: US 2022/0313872 A1
(43) Pub. Date: Oct. 6, 2022

(54) GINGIVAL GRAFT

(71) Applicant: CORNEAT VISION LTD, Raanana (IL)

(72) Inventors: Gilad LITVIN, Moshav Sde Varburg (IL); Almog ALEY-RAZ, Moshav Sde Varburg (IL); Patricia ASSOULINE, KAFAR YONA (IL)

(73) Assignee: CORNEAT VISION LTD, Raanana (IL)

(21) Appl. No.: 17/633,678

(22) PCT Filed: Aug. 12, 2020
(86) PCT No.: PCT/IL2020/050879

§ 371 Date: Feb. 8, 2022

Related U.S. Application Data
(60) Provisional application No. 63/003,319, filed on Apr. 1, 2020.
(60) Provisional application No. 62/885,345, filed on Aug. 12, 2019.

(51) Int. Cl. A61L 27/56; A61L 27/54; A61L 27/26

(52) U.S. Cl. CPC A61L 27/56; A61L 27/54; A61L 27/26; A61L 2400/12; A61L 2430/12

(57) ABSTRACT

The invention provides a synthetic gingival patch graft having a porous polymeric structure and uses thereof in the C method of treatment of periodontal diseases, conditions and symptoms.

### BACKGROUND OF THE INVENTION

Gingival recession, also known as receding gums, is a condition that is manifested by exposure of the roots of the teeth caused by a loss of gum tissue and/or retraction of the gingival margin from the crown of the teeth first down the neck exposing it, then eventually exposing parts of the root. Gum recession is a common typical condition in adults over the age of 40, but it may also occur starting from the age of a teenager, or around the age of 10. It may exist with or without concomitant decrease in crown-to-root ratio (recession of alveolar bone). In most cases, receding gums are a progressive condition that occurs gradually over years. Classification of the condition is based on the position of interdental papilla and buccal/lingual/palatal recessions.

There are many possible causes for gingival recession: (i) the most common cause is gum disease (periodontal disease); (ii) overaggressive brushing and improper flossing (i.e., flossing too roughly or aggressively) which may cut into the gums; (iii) hereditary thin, fragile or insufficient gingival tissue predisposes to gingival recession; (iv) consumption of tobacco products, which affect the mucous membrane lining in the mouth; (v) self-inflicted trauma, such as habits like digging a fingernail or pencil into the gum and piercings in the lip or tongue; (vi) diseases such as: scurvy (lack of dietary vitamin C), acute necrotizing ulcerative gingivitis; (vii) abnormal tooth position, such as tooth crowding, giving inadequate cover of one or more teeth by the jaw bone; (viii) intentional gingival retraction performed during dental surgery procedures.

The following signs and symptoms may indicate gum recession: tooth mobility, dentin hypersensitivity, changes in the teeth appearance (appear longer, roots are exposed and visible, tooth feels notched at the gum line, change in the tooth color, spaces between teeth), cavities below the gum line. If the gum recession is caused by gingivitis, the following symptoms may also be present: puffy, red, or swollen (inflamed) gums, gum bleeding while brushing or flossing, bad breath (halitosis). In some cases, it is the treatment of gingivitis that reveals a gum recession problem, that was previously masked by the gums swelling.

The solutions for gingival recession conditions depend on the shape of the gum recession and the levels of bone around the teeth. Such solutions include regeneration of areas of gum recession with new gum tissue using a variety of gum grafting procedures. These procedures involve repositioning of adjacent gum tissue to cover the recession (called a pedicle graft) or use of a free graft of gingiva with or without a connective tissue graft harvested from the roof of the mouth (called collectively a free gingival graft or a Subepithelial connective tissue graft). Alternatively, a material called acellular dermal matrix (processed donated human skin allograft) may be used as a replacement to connective tissue coming from the patient's own palate.

Recent advances have seen the introduction of platelet derived growth factor (PDGF) infused bone graft material. This material is usually combined with the cellular matrix to form a soft bone paste that is then covered by the allograft. The development of this type of bone and tissue cellular matrix (also known as ortho filler) results in greater osseointegration with the patient's healthy bone and soft tissue. Healing from such procedures requires 2-4 weeks. After a few months the results are evaluated, and, in some cases, the new tissue needs to be reshaped in a further procedure to get an optimal result.

The solutions available for gingival recession have many drawbacks. These solutions require at least two painful, technically demanding and time-consuming dental operations, and are limited by the potential graft's thickness and length. Current solutions, being tissue based, are bio-degradable and according to current professional literature have limited long-term efficiency and stability.

### SUMMARY OF THE INVENTION

Thus, there is a need for providing an effective and stable solution for gingival recession that is on the one hand versatile, making the procedure simple to perform, and on the other hand bio-stable, having an improved and lasting effect.

When referring to a “synthetic gingival patch graft” it should be understood to relate to an implant of synthetic polymeric material used to repair a defect the gingival tissue of a subject. In some embodiments, said patch graft of the invention is in the form of a sheet.

In some embodiments, said synthetic gingival patch graft having a porous polymeric structure, has pores of between about 0.01 microns to 5 microns. In some embodiments a synthetic gingival patch graft having a porous polymeric structure, has pores of 0.01, 0.05, 0.1, 0.5, 1, 1.5, 2, 2.5, 3, 3.5, 4, 4.5, 5 microns.

The invention further provides a synthetic gingival patch graft having a porous polymeric structure with pores of between 5 to 20 microns. In some embodiments, a synthetic gingival patch graft having a porous polymeric structure with pores of 5, 6, 7, 8, 9, 10, 11, 12, 13, 14, 15, 16, 17, 18, 19, 20 microns.

In some embodiments, said synthetic gingival patch graft of the invention is a biocompatible patch. In other embodiments, said synthetic gingival patch graft of the invention is a non-degradable patch. In yet other embodiments said synthetic gingival patch graft is a biodegradable patch.

In some embodiments, said synthetic gingival patch graft of the invention has a thickness of between 100-1000 μm. In other embodiments, said synthetic gingival patch graft of the invention has a thickness of between 10-100 μm. In other embodiments, said synthetic gingival patch graft of the invention has a thickness of between 1000-2500 μm.

In some embodiments, said porous polymeric structure comprises at least one polymer. In other embodiments, said porous polymeric structure comprises nanofibers (in some embodiments, said nanofibers are 500 nm to a few micrometers in thickness).

In some embodiments, said porous polymeric structure comprises at least one porous electrospun polymer. In some other embodiments said porous polymeric structure comprises at least one porous printed polymer (using for example a 3D printing device).

In some further embodiments, said porous polymeric structure comprises at least one polymer selected from polycarbonate, poly(DTE carbonate) polycaprolactone (PCL), polylactic acid (PLA), poly-L-lactic acid (PLLA), Poly(DL-lactide-co-caprolactone, Poly(ethylene-co-vinyl acetate) vinyl acetate, Poly(methyl methacrylate), Poly(propylene carbonate), Poly(vinylidene fluoride), Polyacrylonitrile, Polycaprolactone, Polycarbomethylsilane, Polylactic acid, Polystyrene, Polyvinylpyrrolidone, poly vinyl alcohol (PVA), polyethylene oxide (PEO), polyurethane (including aromatic polyurethane), polyvinyl chloride (PVC), hyaluronic acid (HA), chitosan, alginate, polyhydroxybuyrate and its copolymers, Nylon 11, Cellulose acetate, hydroxyappetite, poly(3-hydroxybutyric acid-co-3-hydroxyvaleric acid), poly(DL-lactide), polycaprolactone, and poly(L-lactide) or any combination thereof

Electrospun fibers are typically several orders in magnitude smaller than those produced using conventional spinning techniques. By optimizing parameters such as: i) the intrinsic properties of the solution including the polarity and surface tension of the solvent, the molecular weight and conformation of the polymer chain, and the viscosity, elasticity, and electrical conductivity of the solution; and ii) the operational conditions such as the strength of electric field, the distance between spinneret and collector, and the feeding rate of the solution, electrospinning is capable of generating fibers as thin as tens of nanometers in diameter. Additional parameters that affect the properties of electrospun fiber include the molecular weight, molecular-weight distribution and structure (branched, linear etc.) of the polymer, solution properties (viscosity, conductivity and surface tension), electric potential, flow rate and concentration, distance between the capillary and collection screen, ambient parameters (temperature, humidity and air velocity in the chamber), motion of target screen (collector) and so forth. Fabrication of highly porous fibers may be achieved by electrospinning the jet directly into a cryogenic liquid. Well-defined pores developed on the surface of each fiber as a result of temperature-induced phase separation between the polymer and the solvent and the evaporation of solvent under a freeze-drying condition.

Several approaches have been developed to organize electrospun fibers into aligned arrays. For example, electrospun fibers can be aligned into a uniaxial array by replacing the single-piece collector with a pair of conductive substrates separated by a void gap. In this case, the nanofibers tend to be stretched across the gap oriented perpendicular to the edges of the electrodes. It was also shown that the paired electrodes could be patterned on an insulating substrate such as quartz or polystyrene so the uniaxially aligned fibers could be stacked layer-by-layer into a 3D lattice. By controlling the electrode pattern and/or the sequence for applying high voltage, it is also possible to generate more complex architectures consisting of well-aligned nanofibers.

Electrospun nanofibers could also be directly deposited on various objects to obtain nanofiber-based constructs with well-defined and controllable shapes. In addition, one can manually process membranes of aligned or randomly oriented nanofibers into various types of constructs after electrospinning: for example, fabrication of a tube by rolling up a fibrous membrane or the preparation of discs with controllable diameters by punching a fibrous membrane.

The present invention relates to any eletrospinning technique known in the art, which includes Electrospinning, J. Stanger, N. Tucker, and M. Staiger, I-Smithers Rapra publishing (UK), An Introduction to Electrospinning and Nanofibers, S. Ramakrishna , K. Fujihara, W-E Teo, World Scientific Publishing Co. Pte Ltd (June 2005), Electrospinning of micro- and nanofibers: fundamentals and applications in separation and filtration processes, Y. Fillatov, A. Budyka, and V. Kirichenko (Trans. D. Letterman), Begell House Inc., New York, USA, 2007, which are all incorporated herein by reference in their entirety.

Suitable electrospinning techniques are disclosed, e.g., in International Patent Application, Publication Nos. WO 2002/049535, WO 2002/049536, WO 2002/049536, WO 2002/049678, WO 2002/074189, WO 2002/074190, WO 2002/074191, WO 2005/032400 and WO 2005/065578, the contents of which are hereby incorporated by reference. It is to be understood that although the according to the presently preferred embodiment of the invention is described with a particular emphasis to the electrospinning technique, it is not intended to limit the scope of the invention to the electrospinning technique. Representative examples of other spinning techniques suitable for the present embodiments include, without limitation, a wet spinning technique, a dry spinning technique, a gel spinning technique, a dispersion spinning technique, a reaction spinning technique or a tack spinning technique. Such and other spinning techniques are known in the art and disclosed, e.g., in U.S. Pat. Nos., 3,737,508, 3,950,478, 3,996,321, 4,189,336, 4,402,900, 4,421,707, 4,431,602, 4,557,732, 4,643,657, 4,804,511, 5,002,474, 5,122,329, 5,387,387, 5,667,743, 6,248,273 and 6,252,031 the contents of which are hereby incorporated by reference.

In some embodiments, said synthetic gingival patch graft of the invention further comprises at least one active agent. In some embodiments, said at least one active agent is selected from a protein, type I collagen, fibronectin, or TGF- beta 2, heparin, growth factors, antibodies, antimetabolites, chemotherapeutic agents, anti-inflammatory agent, anti-biotic agent, and any combinations thereof

In some embodiments, said synthetic gingival patch graft of the invention is cut into a designated shape (in some embodiments, said cut is laser cut, manual cut, pressure cut and so forth).

In some embodiments, said synthetic gingival patch graft of the invention further comprises at least one non-porous layer. In some embodiments, said at least one non-porous layer is in the form of a film. When used as a tissue replacement in periodontal surgery, said at least one non-porous layer or film is placed on the bone side of the cavity to be filled.

When referring to a “non-porous layer” it should be understood to encompass a film layer that has substantially no pores, thus not penetrable, impervious as compared with said porous layer of the gingival graft patch of the invention.

In some embodiments, said non-porous layer is a biocompatible patch. In other embodiments, said non-porous layer is a non-degradable patch. In yet other embodiments said non-porous layer is a biodegradable patch.

In some embodiments, said non-porous layer has a thickness of between 100-1000 μm. In other embodiments, said non-porous layer has a thickness of between 10-100 μm. In other embodiments, said non-porous layer has a thickness of between 1000-2500 μm.

In some embodiments, said non-porous polymeric structure comprises at least one polymer. In other embodiments, said non-porous polymeric structure comprises nanofibers (in some embodiments, said nanofibers are 500 nm to a few micrometers in thickness).

In some further embodiments, said non-porous polymeric structure comprises at least one polymer selected from polycarbonate, poly(DTE carbonate) polycaprolactone (PCL), polylactic acid (PLA), poly-L-lactic acid (PLLA), Poly(DL-lactide-co-caprolactone, Poly(ethylene-co-vinyl acetate) vinyl acetate, Poly(methyl methacrylate), Poly(propylene carbonate), Poly(vinylidene fluoride), Polyacrylonitrile, Polycaprolactone, Polycarbomethylsilane, Polylactic acid, Polystyrene, Polyvinylpyrrolidone, poly vinyl alcohol (PVA), polyethylene oxide (PEO), polyurethane (including aromatic polyurethane), polyvinyl chloride (PVC), hyaluronic acid (HA), chitosan, alginate, polyhydroxybuyrate and its copolymers, Nylon 11, Cellulose acetate, hydroxyappetite, poly(3-hydroxybutyric acid-co-3-hydroxyvaleric acid), poly(DL-lactide), polycaprolactone, and poly(L-lactide) or any combination thereof

When relating to “periodontal disease, condition or symptom” it should be understood to encompass inflammatory conditions affecting the tissues surrounding the teeth such as for example gum disease, gingivitis, periodontitis, tooth decay, tooth loss, bone loss and any combinations thereof

In some embodiments, such periodontal disease, condition or symptom may cause the need for periodontal surgery.

The term “periodontal surgery” is meant to encompass a form of dental surgery that prevents or corrects anatomical, traumatic, developmental, or plaque-induced defects in the bone, gingiva, or alveolar mucosa. The objectives of this surgery include accessibility of instruments to root surface, elimination of inflammation, creation of an oral environment for plaque control, periodontal diseases control, oral hygiene maintenance, maintain proper embrasure space, address gingiva-alveolar mucosa problems, and esthetic improvement. The surgical procedures include among others, crown lengthening, frenectomy, mucogingival flap surgery, gingivectomy, apically repositioned flap (APF) surgery, apically repositioned flap (APF) with osseous reduction (osteoplasty/ostectomy) and any combinations thereof

The invention further provides a synthetic gingival patch graft as disclosed herein above and below for use in periodontal and/ or dental surgery.

The invention further provides a synthetic gingival patch graft as disclosed herein above and below for use in the treatment of periodontal disease, condition or symptom.

The invention further provides a synthetic gingival patch graft as disclosed herein above and below for use in the treatment of gingival recession.

The invention further provides a synthetic gingival patch graft as disclosed herein above and below for use in periodontal root coverage procedure.

The invention further provides a synthetic gingival patch graft as disclosed herein above and below for use in the treatment of periodontal root disease, condition or symptom.

In some embodiments, a synthetic gingival patch graft of the invention is a periodontal tissue replacement patch.

In other embodiments, said synthetic gingival patch graft of the invention is a periodontal tissue supplement patch.

In further embodiments, said synthetic gingival patch graft of the invention is a periodontal tissue reconstruction/regeneration patch.

The invention further provides a device comprising at least one synthetic gingival patch graft of the invention. In some embodiments said device is a dental implant comprising said at least one synthetic gingival patch graft of the invention (for example, covered by said at least one synthetic gingival patch graft of the invention).

The invention further provides a kit comprising at least one synthetic gingival patch graft of the invention, means for its periodontal implanting/placement in the gingival tissue of a subject and instructions for use.

In some embodiments, instructions of use may include: instructions for the care giver how to custom cut the graft patch of the invention, how to place said graft patch invention for example, over the teeth and on the gingival tissue (see for example FIG. 1, 110).

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1E show embodiments of a synthetic gingival patch graft of the invention.

FIGS. 2A-2B show embodiments of a synthetic gingival patch graft of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

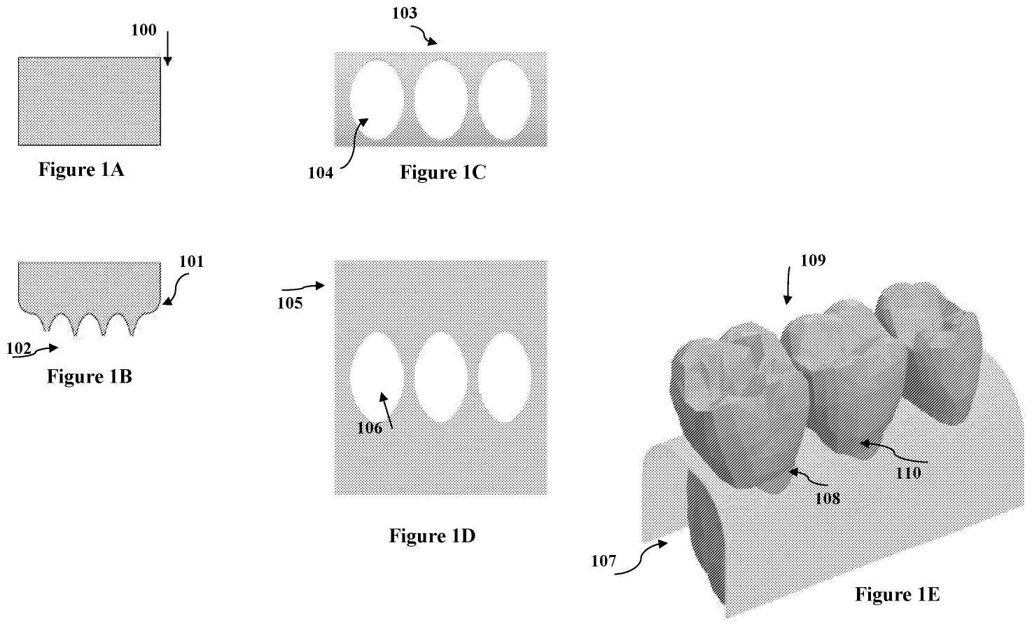

FIG. 1A shows a synthetic gingival patch graft of the invention (100). FIG. 1B shows the synthetic gingival patch graft of the invention (101) with grooves (102) cut in a predetermined shape to be placed periodontally. FIG. 1C and FIG. 1D show embodiments of a synthetic gingival patch graft of the invention (103) and (105) with holes (104) and (106) cut in a predetermined shape to be placed periodontally. FIG. 1E shows a synthetic gingival patch graft of the invention (107), placed on a representative periodontal segment (109), wherein the holes (108) cut into the patch allow the placement over the teeth and on the gingival tissue (110).

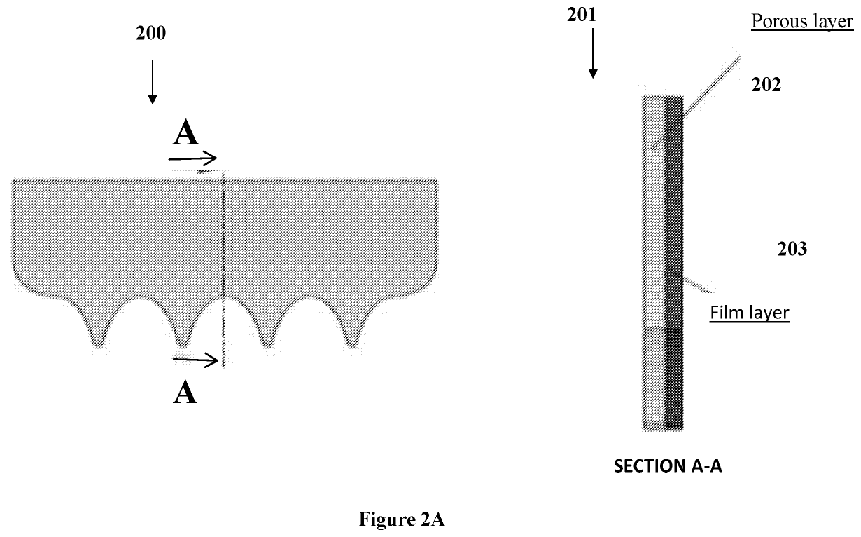

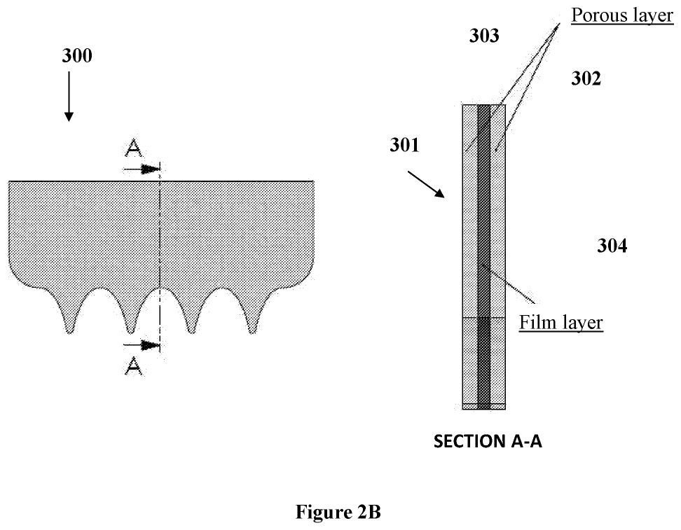

FIG. 2A shows a synthetic gingival patch graft of the invention (200 and 201 the AA cross section of 200) that comprises in addition to the porous layer (202), a non-porous (film) layer (203). FIG. 2B shows synthetic gingival patch graft of the invention (300 and 301 the AA cross section of 300) that comprises two porous layers (302 and 303) and one non-porous (film) layer (304) in between them.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A synthetic gingival patch graft having a porous polymeric structure with pores of less than 5 microns; wherein said patch is a non-degradable patch.

2. A synthetic gingival patch graft according to claim 1, wherein said pores are between about 0.01 microns to about 5 microns.

3. A synthetic gingival patch graft according to claim 1, wherein said pores are between 5 to 20 microns.

4. A synthetic gingival patch graft according to claim 1, being a biocompatible patch.

5. A synthetic gingival patch graft according to claim 1, having a thickness of between 10-100 μm.

6. (canceled)

7. (canceled)

8. A synthetic gingival patch graft according claim 1, wherein said porous polymeric structure comprises at least one polymer.

9. A synthetic gingival patch graft according to any one of the preceding claim 1, wherein said porous polymeric structure comprises nanofibers.

10. A synthetic gingival patch graft according to claim 1, wherein said porous polymeric structure comprises at least one porous electrospun polymer.

11. (canceled)

12. A synthetic gingival patch graft according to claim 1, further comprising at least one active agent.

13. (canceled)

14. A synthetic gingival patch graft according to claim 1, further comprising at least one non-porous layer.

15. A synthetic gingival patch graft according to claim 1, being selected from a tissue replacement patch, a tissue supplement patch and a tissue reconstruction/regeneration patch.

16. (canceled)

17. (canceled)

18. A method of treating at least one periodontal disease, condition or symptom in a subject in need thereof wherein said method comprising implanting a synthetic gingival patch graft having a porous polymeric structure with pores of less than 5 microns; wherein said patch is a non-degradable patch.

19. A method according to claim 18, wherein said at least one periodontal disease, condition or symptom is selected from gingival recession and periodontal rood disease including any conditions and symptoms thereof.

20. (canceled)

21. A device comprising at least one synthetic gingival patch graft having a porous polymeric structure with pores of less than 5 microns; wherein said patch is a non-degradable patch.

22. (canceled)
